(19) United States
(12) Patent Application Publication
(10) Pub. No.: US 2024/0389919 A1
(43) Pub. Date: Nov. 28, 2024

(54) System and Method for Assessing Brain Function Real Time After Brain Injury

(71) Applicant: Rosa Bell, Montgomery, AL (US)

(72) Inventor: Rosa Bell, Montgomery, AL (US)

(21) Appl. No.: 18/789,229

(22) Filed: Jul. 30, 2024

Related U.S. Application Data
(63) Continuation of application No. 16/294,317, filed on Mar. 6, 2019, now abandoned.
(60) Provisional application No. 62/639,294, filed on Mar. 6, 2018.

(51) Int. Cl. A61B 5/291; A61B 5/00

(52) U.S. Cl. CPC A61B 5/291; A61B 5/4064; A61B 5/6803; A61B 5/7225; A61B 5/0006

(57) ABSTRACT

A method, and a system to perform the method, of assessing brain injuries in real time, the method including placing an EEG cap on a subject's head at a first location, processing diagnostic data including brain wave data using a first communication device at the first location, transmitting the diagnostic data to a second communication device located at a remote second location, and displaying the diagnostic data in real time on the second communication device.

### CROSS-REFERENCE TO RELATED APPLICATIONS

This application is a continuation of U.S. patent application Ser. No. 16/294,317, filed on Mar. 6, 2019, which in turn claims the benefit of U.S. Provisional Patent Application Ser. No. 62/639,294, filed on Mar. 6, 2018, each of which is incorporated herein in its entirety by reference.

### FIELD OF INVENTION

The present general inventive concept relates to electroencephalography (EEG) tests, and, more particularly, methods, devices, and systems to perform EEG tests at a remote location in real time.

### BACKGROUND

When dealing with diagnosing head injuries, such as concussions, an examination within the first few minutes after the injury is beneficial both because of the simple fact of getting a faster assessment when needed, and because a quicker diagnosis is more accurate. Because time is of the essence in such a situation, it is particularly valuable to be able to make such an assessment before being moved to facilities that are generally equipped and staffed to conduct such examinations. For example, there are multiple sports in which either intentional or incidental contact can result in possible concussions that should be evaluated at the very least before a player returns to the ongoing game. For example, in football players are having violent contact on almost every play, in baseball players are in danger of being hit in the head with the ball, soccer players routinely collide and strike balls with their heads, and so on. Also, the value of fast examination and assessment is certainly not limited to the field of sports, as such injuries can happen in remote locales to, for example, members of the military. Even medical organizations such as urgent care facilities may not have the personnel and/or equipment to facilitate a head injury examination quickly enough to ensure higher accuracy.

Although such concerns are present in many types of activities and occupations, as one example concussion assessments are a particularly large issue currently in the National Football League (NFL). The NFL has made 47 rule changes since 2002 to protect players, improve practice methods, better educate players and personnel on concussions, and strengthen the league's medical protocols. The NFL deploys 29 medical professionals on the sidelines for each game. With such an arrangement in place, the data of reported concussions from year 2016 indicates that there is a wide gap between teams with the most concussions and the least. For example, over the past few years, the Cincinnati Bengals have averaged 17concussions a year, while the Miami Dolphins have averaged three. (The Sportster-2017) This makes it clear that more objective ways of assessing brain function, concussion, and traumatic brain injury is significantly needed. The large discrepancy in results is very suggestive and is just one of the few examples that prove that there is a need for a more objective way to assess for concussion and traumatic brain injury quickly and objectively, so players brains can be saved.

### BRIEF SUMMARY

According to various example embodiments of the present general inventive concept may provide a method and system of assessing both brain waves and the observable physical attributes and responses of the individual being assessed, quickly after a potential brain injury, and having the data analyzed by trained medical personnel at another location.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a method of assessing brain injuries in real time, the method including placing an EEG cap on a subject's head at a first location, processing diagnostic data including brain wave data using a first communication device at the first location, transmitting the diagnostic data to a second communication device located at a remote second location, and displaying the diagnostic data in real time on the second communication device.

The foregoing and/or other aspects and advantages of the present general inventive concept may also be achieved by a system to assess brain injuries in real time, the system including an EEG cap to be placed on a subject's head at a first location, a first communication device configured to process diagnostic data including brain wave data at the first location, and to transmit the diagnostic data; and a second communication device configured to receive the diagnostic data and display the diagnostic data in real time at a remote second location.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings and photographs, some of which illustrate example embodiments of images that may be displayed on a screen of a processing and/or communication device according to various example embodiments of the present general inventive concept, in which:

FIG. 1 illustrates components of a portable real time brain function assessing system according to an example embodiment of the present general inventive concept;

FIG. 2 illustrates a pack to carry the system of FIG. 1 according to an example embodiment of the present general inventive concept;

FIGS. 3A-3C illustrate various electrode caps to be used with the system of FIG. 1 according to example embodiments of the present general inventive concept;

FIGS. 4A-4D illustrate example communication devices used in the system of FIG. 1 according to example embodiments of the present general inventive concept;

FIG. 5 illustrates an example of brain injury assessment data displayed on various screens communicating with the system of FIG. 1 according to an example embodiment of the present general inventive concept;

FIG. 6 illustrates an example of brain injury assessment data displayed on a smartphone according to an example embodiment of the present general inventive concept;

FIG. 7 illustrates a remotely located doctor reviewing system data according to an example embodiment of the present general inventive concept;

FIG. 8 illustrates a method of assessing a brain injury according to the present general inventive concept; and

FIG. 9 illustrates a block diagram of various components of a portable real time brain function assessing system according to an example embodiment of the present general inventive concept.

### DETAILED DESCRIPTION

Reference will now be made to the example embodiments of the present general inventive concept, examples of which are illustrated in the accompanying drawings, illustrations, and photographs, all of which may be referred to as figures herein. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

Although the example embodiments described herein typically are discussed in terms of a software application operated on a remote dedicated device, it will be understood by those skilled in the art that a host of other example embodiments may be applicable to the present general inventive concept. For example, the methods and/or operations described herein may be performed on a dedicated communication device or may be software implemented by a host of different communication devices such as a laptop computer, smart phone, and so on.

Various example embodiments of the present general inventive concept provide a system and method to assess potential brain trauma at a remote location and provide diagnostic data to a doctor, and/or other medical personnel, at another location for more detailed analysis. Thus, real-time analysis can be available immediately after such an injury. This invention relates to assessing seamless, real time Electroencephalographic (EEG) objective functional brain changes in brain injuries immediately to assess for functional objective changes in the brain. Specifically, the present general inventive concept relates to a method and system of assessing this information in real time, in a way that is objective. Additionally, the present general inventive concept provides real time communication, both visually and verbally, between the subject and the observer and interpreter of the brainwave data. This can be crucial in any setting in which brain function needs to be accessed objectively, and immediately. This can involve any genre where there is transient alteration of consciousness and there is a possible shearing force of brain in skull. As previously discussed, NFL football games are one glaring example in which such a method is needed. As players' verbal answers may be tainted by their desire to return to the field, and because it is very expensive to have medical doctors on site simply to accurately and objectively diagnose injuries such as concussions, there are obviously many reasons to have a doctor at a location other than the field to be able to receive brain activity information from a technician trained with assessment equipment, so as to be able to able diagnose problems in a more honest, and less expensive, way.

The NFL Player Concussion Pamphlet lists several common symptoms of concussions, including imbalance, headache, confusion, memory loss, loss of consciousness, vision change, hearing change, mood change, fatigue, and malaise. Studies suggest that over 60% of players, after a hit on the field and possible concussion, do not have many or any of these symptoms. Also, many of these symptoms may go unnoticed, and therefore concussions are not properly assessed. Various example embodiments of the present general inventive concept in which objective brain data is assessed in real time could alleviate at least some of this discrepancy.

Various example embodiments of the present general inventive concept provide a method and system of assessing both brain waves and the observable physical attributes and responses of the individual being assessed. This may be done quickly and seamlessly after any disruption in brain function, such as a concussion or potential concussion, in real time through a locally located technician placing an electrode cap on the injured individual and using the system to interact with the injured individual, such that a doctor at a remote location can monitor the EEG data from the electrode cap, see the actual physical responses from the injured individual, hear the individual's responses, and also interact with the individual through additional follow-up queries and observations to gain more information. It will be recognized that, as used herein, the term “interact” may include, but is not necessarily limited to, the locally located technician posing a series of queries aimed at assessing whether the individual exhibits at least one of the above-described symptoms indicative of concussion and observing the injured individual's responses. Diagnosis as soon as possible after a brain injury can be crucial to the accuracy of the diagnosis.

Various example embodiments of the present general inventive concept may provide a portable compact EEG machine in electrical communication with a processing and communication device such as a portable computer which may have a battery backup system. Various example embodiments of the present general inventive concept may include an electrode placement amplifier, and/or electrode caps having various different configurations based on the head type, hair type, etc., of the injured party. Installation of the electrode cap may be administered according to the 10/20 electrode placement system which is the worldwide system for assessing brainwaves via EEG.

In various example embodiments of the present general inventive concept, after quickly placing the electrode cap on the injured party, brain waves can be promptly viewed via any type of communication devices such as a console computer, handheld device, laptop computer, smart phone, etc., via protected and encrypted data files, to look at the data corresponding to the brain waves. The data can be viewed both locally by the technician, and by a doctor or other such trained medical professional at a remote location. The system may include a camera to capture images of the injured party such that physical and verbal responses or activities may be viewed by the medical personnel to aid in the diagnosis of the injury. Thus, various brain wave and other such data can be viewed both locally and remotely and interpreted in real time. Using the computer, electrode cap, and camera, both the local technician and the remote doctor are able to interpret data in real time and communicate both verbally and visually with each other as well as with the individual with the injury. The electrical communications to allow such a process may be by any host of configurations and/or protocols, such as by a wired connection, wi-fi, Bluetooth, etc. Any of the acquired data may be stored for later viewing, by storing the data locally at the communication device, remotely at the doctor's location, and/or by storing the data wirelessly in “the cloud.” It is noted that in the descriptions herein, the injured party being diagnoses for a head injury may be alternately referred to as the patient, subject, injured individual, and so on.

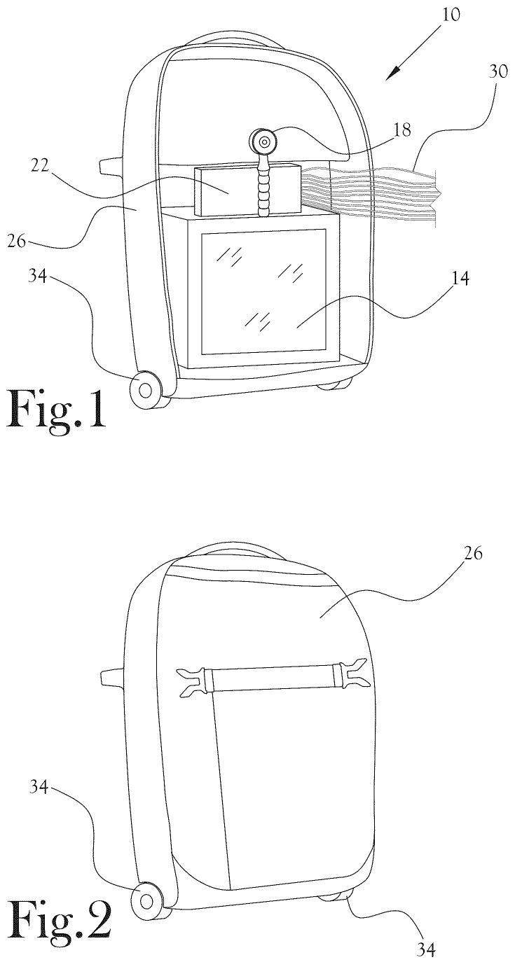

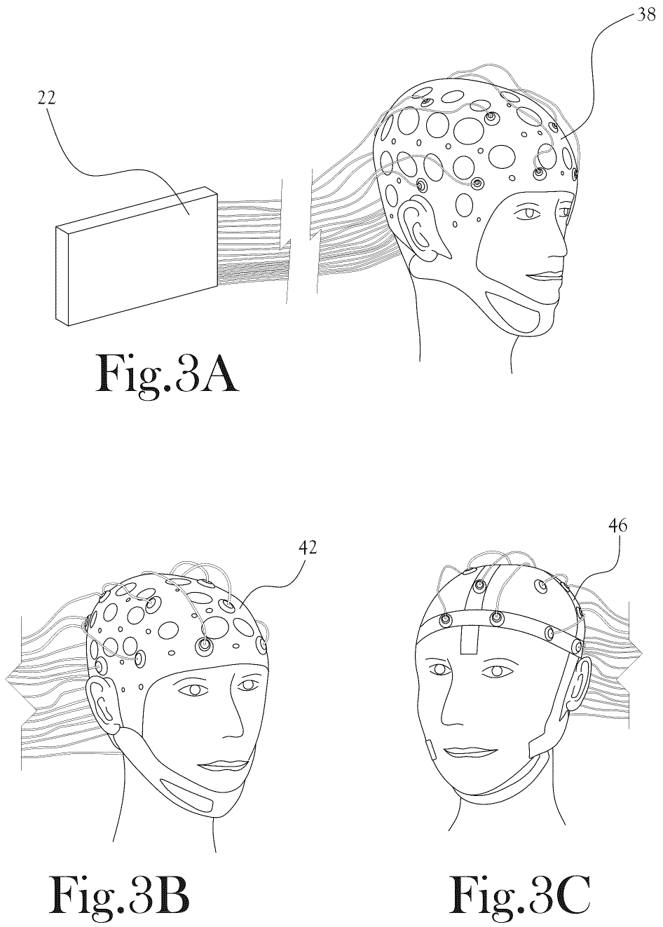

FIG. 1 illustrates components of a portable real time brain function assessing system 10 according to an example embodiment of the present general inventive concept. The example embodiment illustrated in FIG. 1 includes a computer 14 to compile brain assessment data collected from the subject, or patient, a camera 18 or other image capturing device to allow a remotely located doctor to see and hear patient reactions and responses, and an amplifier 22 to amplify electrode signals gathered from an electrode cap worn by the patient. The computer 14 may be a dedicated device constructed for the portable real time brain function assessing system 10, or may be a conventional device (laptop, tablet, etc.) loaded with appropriate software to operate the system, as well as with wireless communication capability to transmit assessment data to and from the remotely located doctor. The components of the system 10 illustrated in FIG. 1 are arranged in a portable carrying pack 26, illustrated in FIG. 1 without the front portion for clarity, to provide easy and convenient mobility for the system 10. It is understood that various other example embodiments of the present general inventive concept may include fewer or more components, and in a variety of configurations that may differ from the example illustrated in FIG. 1 without departing from the scope of the present general inventive concept. A few examples of electrode caps that may be used with the system 10 will be illustrated herein in FIG. 3. Example embodiments of the camera 18 may typically have a built-in microphone so that both visual and audio data may be transmitted to the remote doctor. As illustrated in FIG. 1, a plurality of electrical leads 30 are provided to the system 10 to receive data from the electrode cap on the patient. Although the leads 30 are illustrated as being connected directly to the amplifier 22 in the example embodiment illustrated in FIG. 1, various other example embodiments may include different arrangements and points of connection with the computer 30 and overall system 10. For example, in various example embodiments the leads 30 may be connected to a transmitter to transmit the signals from the electrode cap to a remote location. A speaker may also be provided to the system to make such communication easier between the doctor and patient or medical assistant working with the patient. It is crucial to diagnose such a head injury within the first 15 minutes in order to obtain more accurate data. Therefore, as soon as possible after the potential injury, for example, as soon as an injured football player makes it back to the sidelines, a technician is able to place the electrode cap on the player and begin the diagnosis procedure. The technician can contact the doctor or other such medical professional immediately before beginning the procedure, so that the doctor may view all of the data. By the computer 14 of FIG. 1 accumulating and communicating the brain wave data in real time and allowing the doctor to interact with the both the technician and the player/subject/patient, the doctor can remotely control the examination and see immediate results, which will greatly aid a more accurate diagnosis.

FIG. 2 illustrates a pack 26 to carry the system of FIG. 1 according to an example embodiment of the present general inventive concept. As illustrated in FIG. 2, the pack 26 may be a compact bag provided with wheels 34, with all the system's equipment enclosed and easily portable to different stadiums, different locations on the sidelines, etc. Various example embodiments of the pack 26 may be configured as a backpack, briefcase, etc., without departing from the scope of the present general inventive concept. In various example embodiments, the system 10 components enclosed in the pack 26 may be situated as illustrated in FIG. 1, such that the system is readily available to operate by simply opening up a zippered or otherwise easily opened panel, flap, or the sort of the pack 26 (not shown in FIG. 1). The pack 26 may have an electrical port (not shown) to provide power to the system 10 in a situation in which a provided battery has exhausted its charge, or to charge the battery, and so on. The pack 26 may also accommodate a power strip to provide power to the components of the system 10.

FIGS. 3A-3C illustrate various electrode caps to be used with the system of FIG. 1 according to example embodiments of the present general inventive concept. FIGS. 3A, 3B, and 3C respectively illustrate different various example configurations 38,42,46 that can be implemented for different head and hair types using the 10/20 international system placement. For example, various example embodiments similar to the illustrations in FIGS. 3A-3B may have openings in the cap to accommodate dreadlocks, so that the electrodes may have a closer proximity to the subject's scalp. A plurality of different electrode caps that may be easily configured by moving the cap and/or diode connections may be included in the system, or a single cap that is easily adjustable through placement and/or reconfiguration of the electrodes may be included. As illustrated in FIG. 3A, the electrodes of the electrode caps may be in electrical communication with the amplifier 22 of the system 10 through the leads 30 illustrated in FIG. 1. In the example embodiments illustrated in FIGS. 3A-3C, the system 10 employs 19 leads 30 from the electrode caps to the amplifier 22, but in other various example embodiments different quantities and/or configurations of leads may be used.

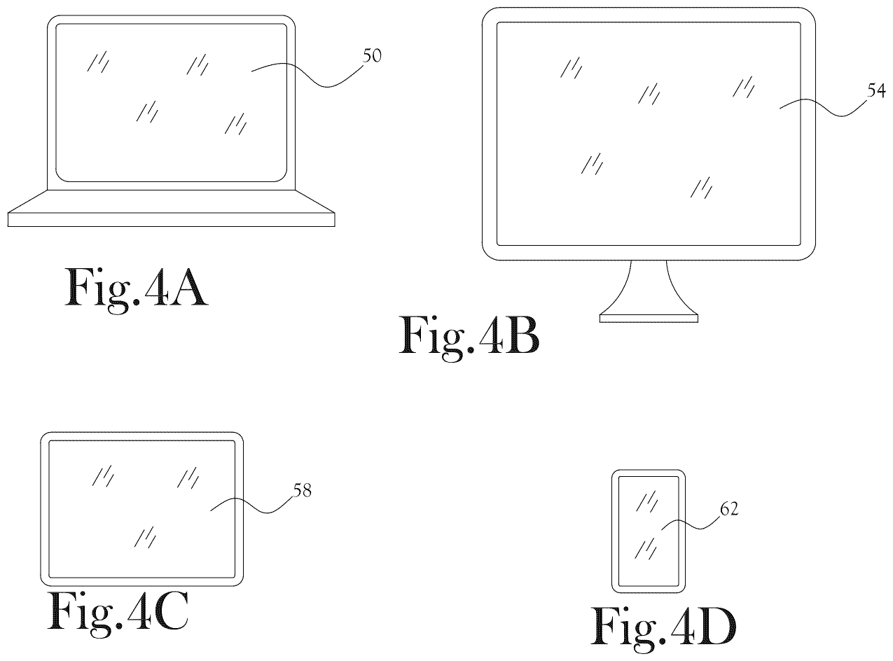

FIGS. 4A-4D illustrate example communication devices that may be used in the system 10 of FIG. 1 according to example embodiments of the present general inventive concept. As illustrated in FIGS. 4A-4D, a laptop computer 50, a desktop computer 54, a handheld device such as a tablet 58, and a smart phone 62 are possible embodiments of communication and processing devices which may be used both at the injury site and at the remote location at which the doctor is performing the diagnosis. By utilizing such devices, the remotely located doctor is able to receive electrode cap data, visual data, and audio data so that a fast and accurate diagnosis can be made for the injured patient. It is noted that the devices illustrated in FIGS. 4A-4D are not an exhaustive list of the communication devices that may be used with the system of the present general inventive concept, as any device with a screen and the ability to communicate and view data from a subject whose brainwaves are being assessed, as well as from the person applying the electrode cap and operating the system locally, is able to be used for remote viewing of the data and the subject, as well as communicating with both other parties in real time.

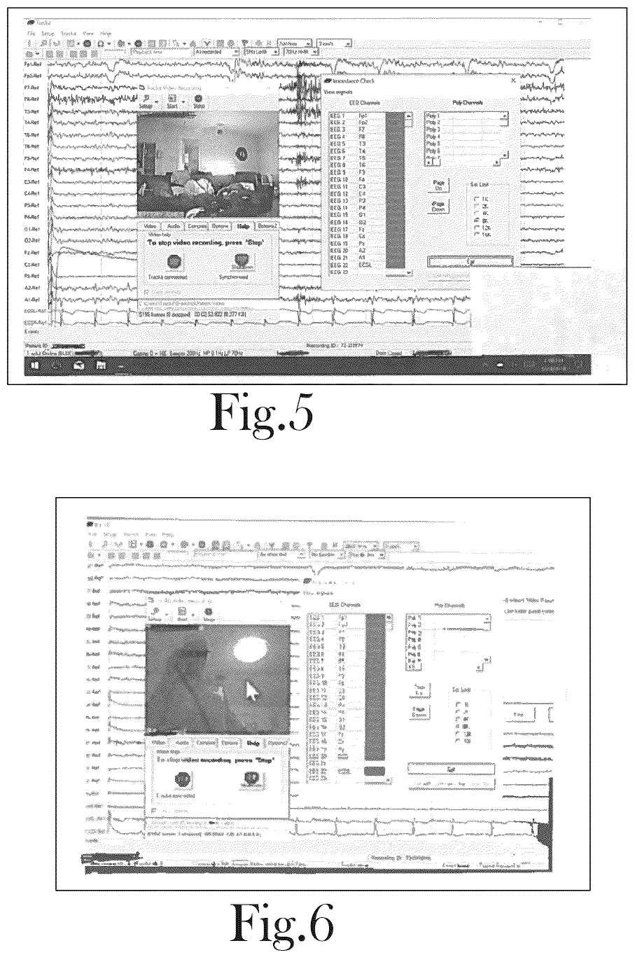

FIG. 5 illustrates an example of brain injury assessment data displayed on various screens communicating with the system 10 of FIG. 1 according to an example embodiment of the present general inventive concept. As illustrated in FIG. 5, the doctor at the remote location is able to see brainwave data, see and hear the subject with the brain injury, and see and hear the technician according to placement of the camera by the technician. In various example embodiments, a camera 18 equipped with microphone may have a handheld configuration for easy manipulation by the technician. The speaker used for communication with the doctor at the remote location may also be provided on the handheld configuration. In various example embodiments, audio transmissions from the doctor to the technician may be routed through an included ear piece (not shown) to exclude the injured subject from hearing various communications. In various example embodiments, the remotely located doctor may be able to manually switch between the handheld or otherwise system speaker and the private line to the technician. FIG. 6 illustrates an example of how such brain injury assessment data may be displayed on a smartphone according to an example embodiment of the present general inventive concept.

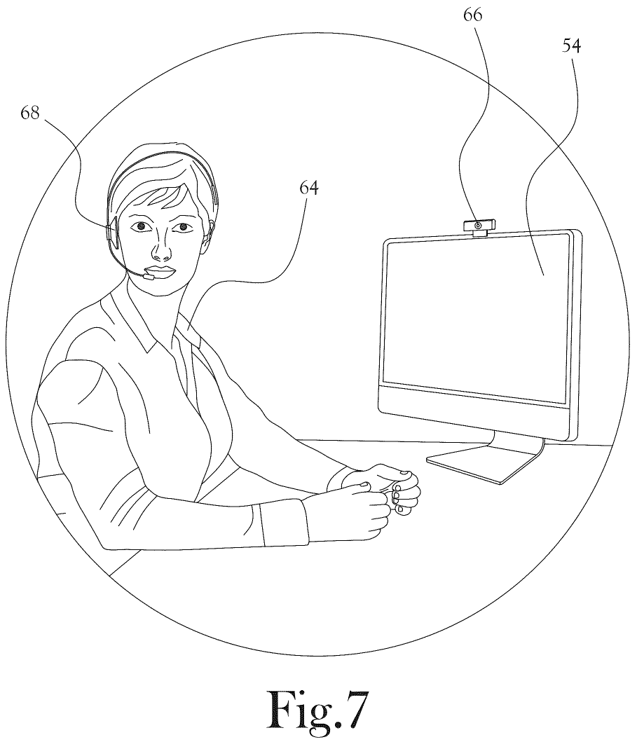

FIG. 7 illustrates a remotely located doctor 64, or other sufficiently trained medical personnel, reviewing system 10 data according to an example embodiment of the present general inventive concept. The doctor 64 may be located at this location and supply interactive diagnostic communications to several other locations. In the example embodiment illustrated in FIG. 7, the doctor 64 is receiving and communicating data through a desktop computer 54 by using a camera 66 and headset 68 in communication with the desktop computer 54. At that remote location, the doctor 64 is able to review brainwave data of actual individual brainwaves being currently assessed, and communicate with the technician applying the electrode cap and potentially applying one or more tests, all in real time while communicating with the injured party and technician. In other various example embodiments, the doctor 64 may be using a portable device such as a laptop 50, tablet 58, or smart phone 62 so that the doctor is not required to be in any one single location to interact with the patient and/or technician using the system 10.

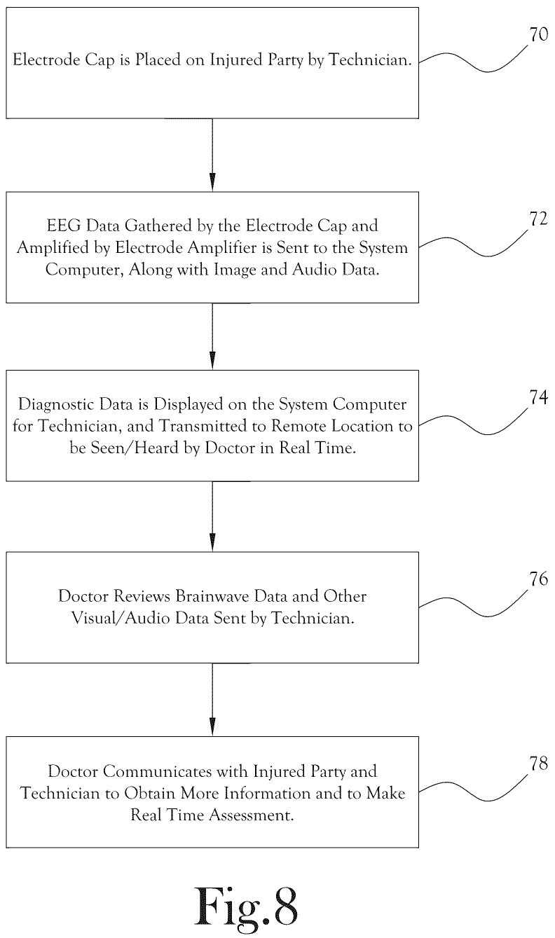

FIG. 8 illustrates a method of assessing a brain injury according to the present general inventive concept. As illustrated in this flowchart, in operation 70 the local technician may place an electrode cap (such as those illustrated in FIGS. 3A-C) on the head of the injured party according to the 10/20 international system placement. In operation 72 EEG data gathered by the electrode cap is amplified by the electrode amplifier and sent to the computer of the portable system, along with images captured by the camera and audio captured by the microphone. In various example embodiments, audio is not employed, and all transmitted data is simply visual. In operation 74, the diagnostic data is able to be seen by the local technician, and is also transmitted to the remote doctor to be seen, and in various example embodiments heard, at the remote location, using any type of communication device with a display screen, such as a PC, laptop, handheld device, smartphone, etc. In operation 76, the doctor at the remote location is able to see the brainwave data and the injured party being assessed, and in operation 78 the doctor is able to communicate with the injured party and the individual administering the tests to guide the tests to get more information in order to make a real time assessment.

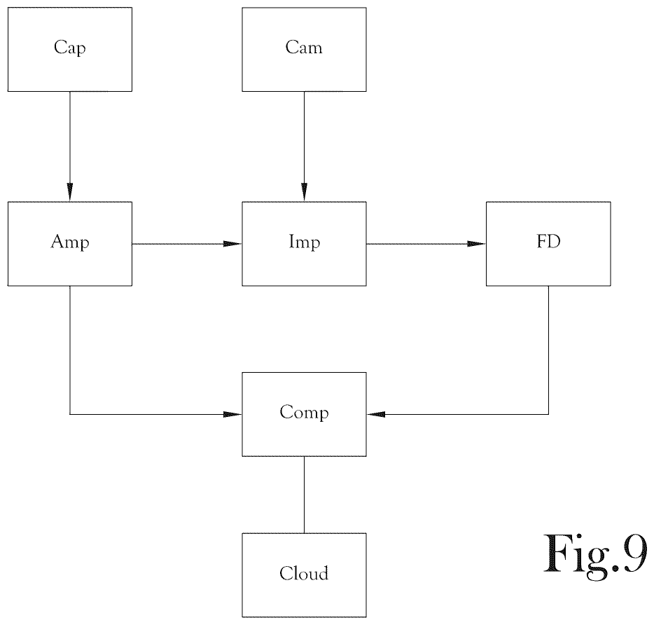

FIG. 9 illustrates a block diagram of various components of a portable real time brain function assessing system according to an example embodiment of the present general inventive concept. Various example embodiments of the portable real time brain function assessing system according to the example embodiment of the present general inventive concept illustrated in FIG. 9 may include the electrode cap placed on the head of the injured individual that transmits brainwave signals from the electrodes provided thereon to an electrode placement amplifier, which then transmits the amplified electrode signals to a local computer. A camera is used to record the activity of the injured party, including responses to any tests that the injured party is asked to complete, such as answering basic cognitive questions. It will be recognized that these “tests” and “cognitive questions” may take the form of a first set of queries posed by the local technician at the sideline of a sporting activity during and/or after placing the electrode cap on the injured party's head. Signals from the camera are received by the computer, which combines the electrode signal information and the camera information and may store the diagnostic information on a flash drive or other such memory storage. The computer may be configured to deliver the diagnostic information to “the cloud” so as to be accessible in real time by a doctor at a remote location. The local technician can receive communication from the doctor through cloud communication, including instructions to perform more tests, to adjust one or more aspects of the testing equipment, and so on. The doctor can also communicate in real time with the patient, to ask him/her further questions and/or to perform other types of tests. The system may be configured to be compact and portable to easily be carried by a single person in a backpack or similar carrier. The system may be provided with a rechargeable battery power system that is configured to carry a large enough charge to perform several such tests over several hours. The camera may be provided with auxiliary lights to allow the locally located technician to apply extra light to the patient for basic or additional tests requested by the doctor through the communication devices. A speaker may be provided to the system through which the remotely located doctor may communicate with the system. Although the block diagram of FIG. 9 shows the data being communicated to the cloud, in various example embodiments the information may be sent directly to the remotely located doctor by any of a number of ways, such as by direct cellular communication, etc.

According to various example embodiments of the present general inventive concept, a communication device with a display and user interface will perform operations according to a machine readable program to prompt a user at a first location to record EEG and visual data, and transmit the data to a remote location to be displayed to a doctor. The operations may also include the doctor communicating with the user and the injured party to obtain further diagnostic information.

Various example embodiments of the present general inventive concept may provide a method of assessing brain injuries in real time, the method including placing an EEG cap on a subject's head at a first location, processing diagnostic data including brain wave data using a first communication device at the first location, transmitting the diagnostic data to a second communication device located at a remote second location, and displaying the diagnostic data in real time on the second communication device. The diagnostic data may also include video data of the subject recorded by a camera. The diagnostic data may also include audio data of the subject recorded by a microphone. The method may further include a user at the first location posing a first series of questions to the subject, recording and/or transmitting the subject's responses to the doctor, and communicating instructions from the doctor at the remote second location to the user at the first location to perform additional diagnostic tests. The additional diagnostic tests may include asking the subject a second series of questions. A technician at the first location may place the EEG cap on the subject's head and operates the first communication device. The method may further include the doctor at the remote second location selectively switching outgoing communications between the technician and the subject. Instructions selected to be delivered only to the technician may be delivered through an earpiece worn by the technician. Instructions selected to be delivered only to the technician may be delivered visually on a screen configured to be seen only by the technician.

Various example embodiments of the present general inventive concept may provide a system to assess brain injuries in real time, the system including an EEG cap to be placed on a subject's head at a first location, a first communication device configured be in electrical communication with the EEG cap and to process diagnostic data including brain wave data at the first location, and to transmit the diagnostic data, and a second communication device configured to receive the diagnostic data and display the diagnostic data in real time at a remote second location. The system may further include a camera to record the subject to provide additional video diagnostic data. The system may further include a microphone to record the subject to provide additional audio diagnostic data. The system may further include a speaker to communicate instructions from the doctor at the remoted second location to the user at the first location to perform additional diagnostic tests. The system may further include a private speaker configured to be heard only by a technician performing diagnostic tests on the subject. The system may further include a packaging to encase the system so as to be handheld and portable for a single user. The system may further include a plurality of EEG caps to configured to be selectively applied according to physical characteristics of the subject.

As a non-exhaustive illustration only, a communication terminal/device/unit described herein may refer to mobile devices such as a cellular phone, a personal digital assistant (PDA), a digital camera, a portable game console, and an MP3 player, a portable/personal multimedia player (PMP), a handheld e-book, a portable lab-top PC, a global positioning system (GPS) navigation, and devices such as a desktop PC, a high definition television (HDTV), an optical disc player, a setup box, and the like capable of wireless communication or network communication consistent with that disclosed herein.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering judgment. Numerous variations, modification, and additional embodiments are possible, and, accordingly, all such variations, modifications, and embodiments are to be regarded as being within the spirit and scope of the present general inventive concept.

Various example embodiments of the present general inventive concept described herein may include operations performed by one or more processors, computers, etc., that are caused to perform these operations by instructions recorded on a non-transitory computer readable storage medium. Various ones of the operations and processes described and/or associated with the described various operations and processes may be performed on any of a host of devices, such as an online server, personal computer, smart phone, tablet computer, etc., or any device containing one or more processors to process such instructions.

Program instructions to perform a method described herein, or one or more operations thereof, may be recorded, stored, or fixed in one or more non-transitory computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein can be easily construed by programmers skilled in the art to which the embodiments pertain based on and using the flow diagrams and their corresponding descriptions as provided herein. Also, the described unit to perform an operation or a method may be hardware, software, or some combination of hardware and software. For example, the unit may be a software package running on a computer or the computer on which that software is running.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings Additional modifications will readily appear to those skilled in the art. Accordingly, departures may be made from such details without departing from the spirit or scope of applicant's general inventive concept.

1. A method of assessing whether an activity participant should return to an activity following a traumatic head injury at an injury site, the method comprising:
relocating the activity participant from an injury site within an activity field to a sideline adjacent the activity field;
at the sideline, a technician placing an EEG cap from a portable carrying pack containing the EEG cap, an EEG signal amplifier, a camera, a microphone, and a first communication device comprising a computer and a transmitter, on the activity participant's head;
at the sideline during and after placing the EEG cap on the activity participant's head, the technician posing a first plurality of queries to the activity participant and observing the activity participant's behavior and responses to the first plurality of queries to assess whether the activity participant exhibits at least one of a group of symptoms indicative of traumatic head injury comprising imbalance, headache, confusion, memory loss, loss of consciousness, vision change, hearing change mood change, fatigue, and malaise;
at the sideline, collecting brain assessment data using the EEG cap, the EEG signal amplifier, the camera, and the microphone, and sending the brain assessment data to the computer, the brain assessment data comprising brain wave data, audio of the subject, video of the head of the subject, and audio of the technician;
at the sideline, processing the brain assessment data including the brain wave data using the first communication device to compile diagnostic data of the subject comprising brain wave data, audio of the subject, video of the head of the subject, and audio of the technician comprising at least one observation of the activity participant by the technician used to assess whether the activity participant exhibits at least one of the group of symptoms indicative of traumatic head injury;
transmitting the diagnostic data from the sideline to a second communication device located at a remote location, the second communication device comprising a microphone and a display device for displaying the brain wave data, audio of the subject, video of the head of the subject, and audio of the technician;
at the remote location, displaying the diagnostic data in real time on the second communication device;
conducting a first communication between a physician at the remote location, the technician at the sideline, and the activity participant at the sideline, in which a second plurality of queries are posed to the activity participant by the physician while the activity participant is directly observed by the technician at the sideline and indirectly observed by the physician using the camera and the second communication device;
conducting a second communication between the physician at the remote location and the technician at the sideline, but excluding the activity participant, in which a third plurality of queries are posed to the technician by the physician regarding observation of the activity participant by the technician at the sideline during the first and second plurality of queries;
at the remote location, the physician assessing brain injuries based on the diagnostic data displayed on the second communication device, the technician's observations during the first and second plurality of queries, and the activity participant's responses to the second queries;
at the remote location, based on the physician's assessment, determining whether the activity participant should return to the activity; and
transmitting a recommendation from the remote location to the sideline as to whether the activity participant should return to the activity;
wherein the operations of relocating the activity participant, placing the EEG cap on the activity participant's head, posing the first plurality of queries and observing the activity participant's behavior and responses, collecting the brain assessment data, processing the brain assessment data, and transmitting the diagnostic data all occur within fifteen minutes after the traumatic head injury.

2. The method of claim 1, wherein the operation of assessing brain injuries further comprises the steps of:
a. from the remote location, asking the activity participant at the sideline a series of questions; and
b. from the sideline, performing a diagnostic test on the activity participant.

3. The method of claim 2, further comprising the operation of:
selectively switching transmission of communication from the remote location between a first mode, in which communication is transmitted to both the technician and the activity participant, and a second mode, in which communication is transmitted to the technician but not the activity participant.

4. The method of claim 3, wherein communication selected to be delivered to the technician through the second mode are delivered through an earpiece worn by the technician.

5. The method of claim 3, wherein communication selected to be delivered to the technician through the second mode is delivered visually on a screen provided by the first communication device that is configured to be seen by the technician.
